(19) United States
(12) Patent Application Publication
(10) Pub. No.: US 2011/0130464 A1
(43) Pub. Date: Jun. 2, 2011

(54) Combination Agent for Improving Carcass Performance in Finishing Pigs

(75) Inventors: Patrick Monahan, Columbia, MD (US); Stephen Hiu, Columbia, MD (US)

(73) Assignee: IGENE BIOTECHNOLOGY, INC., Columbia, MD (US)

(21) Appl. No.: 13/056,650

(22) PCT Filed: Jul. 20, 2009
(86) PCT No.: PCT/US09/51161

§ 371 Date: Feb. 6, 2011

Related U.S. Application Data
(60) Provisional application No. 61/084,625, filed on Jul. 30, 2008.

(51) Int. Cl. A61K 31/137; A61P 3/00

(52) U.S. Cl. 514/653

(57) ABSTRACT

A composition of ractopamine and astaxanthin, included as additives in a feed, for finishing hogs, improves carcass performance.

### BACKGROUND

1. Field of the Invention

The present invention relates to an agent that improves carcass characteristics of late finishing pigs. More specifically, the present invention relates to the addition to the diet of finishing pigs, astaxanthin in combination with Paylean® (ractopamine or ractopamine hydrochloride) as a feed additive to decrease, for example, back fat accumulation, to decrease hack fat depth, to improve percent of standardized fat-free lean, and to improve loin eye area in the carcass of finishing pigs.

2. Background of the Invention

The production of swine (also known as pigs or hogs) for food use is an important industry in the United States with more than 100 million pigs produced each year. Because margins are low, there is demand among producers for agents that increase productivity (known generically as performance), such as by increasing the proportion of meat per carcass, increasing loin eye area, decreasing the amount of back fat, or which provide other beneficial characteristics, such as improved color measurements.

Ractopamine or ractopamine hydrochloride is used as a feed additive in pork production under the trademark Paylean®. It is a β-adrenergic receptor (BAR) agonist which is used to modify growth rate and body composition. As such, its effect in finishing pigs is to take energy from fat growth and redirect that energy to promote the increase of muscle fiber diameter and the growth of lean muscle protein. Paylean® has been shown to increase hot carcass weight, dressing percent and loin eye area in pigs (Trapp et al., Anim. Sci. 80(Suppl 1)104, 2002). The compound finds maximal effect when added to a feed at the rate of 9 or 10 parts per million (ppm).

Astaxanthin (3,3′-dihydroxy-β,β-carotene-4,4′ dione) is the carotenoid that imparts the pink pigment to eggs, flesh, filets, and skin of shrimp, salmon and trout. Astaxanthin belongs to a group of pigments known as carotenoids which exhibit antioxidant properties. Few animals synthesize astaxanthin with most relying on food intake as the source of astaxanthin. Crustaceans, such as crabs, lobsters, crawfish and shrimp obtain the characteristic hue thereof from astaxanthin obtained from food, whether farm-raised or harvested in the wild. Although astaxanthin has been known to color the flesh of salmon and trout, as this compound is a normal part of the diet, it has not previously been known that astaxanthin has the ability to affect color characteristics of the meat of mammals.

Astaxanthin can be synthesized or obtained from natural sources, such as yeast, such as Phaffia rhodozyma (Xanthophyllomyces dendrorhous), from certain algae, such as Hematococcus pluvialis, or from certain bacteria, such as certain Paracoccus spp.

Johnson et al. reported that the astaxanthin from broken Phaffia rhodozyma was deposited in the egg yolks of chicken fed over a 16-day regime (1980, Poultry Sci. 59:1777-1782). Studies have also shown that the presence of astaxanthin in chicken feed increased fertility, weight gain and feed utilization (Yang et al., 2006, Asian-Aust. J. Anim. Sci. 19(7): 1-7). In addition, Phaffia rhodozyma is commonly used, and is commercially available, as a source of astaxanthin for the pigmentation of farm-raised salmonids (Johnson & An, 1991, Crit. Rev. Biotechnology 11(4):297-326).

Certain strains of Phaffia have been manipulated to contain and to produce enhanced levels of astaxanthin, see, for example, U.S. Pat. Nos. 5,182,208 and 5,356,809. Thus, certain strains of Phaffia contain more than 500 parts per million (ppm) astaxanthin per gram dry weight of yeast, more than 600 ppm, more than 700 ppm, more than 800 ppm, more than 900 ppm, more than 1000 ppm, more than 1100 ppm, more than 1200 ppm, more than 1300 ppm, more than 1400 ppm, more than 1500 ppm, more than 1600 ppm, more than 1700 ppm, more than 1800 ppm, more than 1900 ppm, more than 2000 ppm, more than 5000 ppm or more astaxanthin per dry gram of yeast. Sources providing greater amounts of astaxanthin provide an economic advantage.

Dietary carotenoids, including astaxanthin, are used as a nutraceutical supplement. Dietary carotenoids are asserted to prevent infections. Astaxanthin, a nonprovitamin α-carotenoid is as active, and at times more active than β-carotene in enhancing immune response in animals and humans (Chew & Park, 2004, J. Nutr. 134:257S-261S). Astaxanthin has also been described in a composition comprising a source of long chain polyunsaturated fatty acid and a carotenoid for prophylactic and/or therapeutic use in the healing of trauma-induced and stress-induced inflammatory conditions (PCT Pub. No. WO2004/112776).

For animal use, astaxanthin was used as a feed ingredient in combination with L-ascorbic acid derivatives as an anti-stress composition for animals (U.S. Pat. No. 5,937,790). In another application, astaxanthin is claimed to suppress body fat gain in mammals where the mammal is a human, based on a study done in mice fed high doses of astaxanthin as part of a high-fat diet (U.S. Publ. No. 2007129436).

In the swine production industry, astaxanthin from astaxanthin-rich algae meal is alleged to improve sow and litter performance when added as an ingredient incorporated into normal diet when fed before farrowing, during lactation and after weaning (Inborr et al., 1997, Proceedings of the 7^{th} International Symposium on Digestive Physiology in Pigs, 26-28 May). The mechanism for this improvement was thought to be the result of an improvement in immune response of the animals fed astaxanthin. U.S. Pat. No. 6,054,491 teaches addition of 5 ppm astaxanthin in the feed improved performance of sows by reducing the number of stillborn in the litter and improving parity. Yang et al., supra, showed that addition of 0, 1.5 and 3.0 ppm astaxanthin incorporated into the diet of finishing pigs had no significant effect on production performance, but that there was a linear effect on dressing percentage, back fat thickness and loin muscle area with increasing dietary astaxanthin level.

The reasons for the improved carcass traits and meat quality when astaxanthin-enriched feed is used are not known.

The present invention provides a composition consisting of astaxanthin, preferably from a natural source, such as Phaffia rhodozyma yeast, which when combined with Paylean® enhances carcass characteristics in finishing pigs at rates or amounts better or greater than when either active agent is used alone.

### DESCRIPTION OF THE INVENTION

The present invention provides a composition for improving carcass traits and meat quality of finishing pigs (or hogs or swine, as used in the domesticated or agricultural trade) destined for slaughter. Astaxanthin is provided in the diet. Astaxanthin can be produced by Phaffia rhodozyma. The astaxanthin according to the invention can be obtained from other sources including synthetic astaxanthin and other naturally-produced astaxanthin, such as from bacteria or algae. The astaxanthin is used in combination with Paylean® to enhance certain carcass traits and meat quality of finishing pigs.

In the agricultural arts, such finishing pigs or hogs generally include growing females (gilts) and growing castrated males (barrows) that are being fed for slaughter. Typically, pigs are classified by weight and/or age. Hence, piglets are animals from birth to weaning, generally from 14 to 35 days of age: nursery pigs are animals from weaning to about 70 days of age; growing pigs are animals from about 70 days of age to about 125 days of age, where they generally weigh about 160 lbs.; and finishing pigs, which are animals from about 125 days of age to about 190 days of age, where generally the market weight of a hog is about 270 lbs. That is about the time the pigs reach sexual maturity.

For the purposes of the instant invention, the nutrition composition of interest generally is provided to pigs destined for slaughter, the finishing hogs, generally by way of additives to feed. However, the nutrition composition of interest can be used for pigs of any age, any stage of development or any weight, and can be administered by any known means.

The source of natural astaxanthin used in this invention is the yeast Phaffia rhodozyma. The isolated astaxanthin is commercially available under the trade name Aquasta®. Production of this yeast is described in U.S. Pat. Nos. 5,356,809 and 5,182,208.

Paylean® was obtained from Elanco Animal Health and used as recommended by the manufacturer.

The present invention further provides a method of simultaneously decreasing backfat while increasing fat-free lean percent and improving meat color in pigs. In a preferred embodiment of said method according to the invention, astaxanthin, which is obtained from Phaffia rhodozyma, is administered to the diet of pigs ideally in the range of from about 3.5 to about 10 mg agent per kg feed.

The astaxanthin can be used in a variety of ways for ingestion by hogs. Thus, the astaxanthin can be an ingredient in a mixture, can be part of a feed, added to a feed, a component of a supplement, can be part of a food, added to a food and the like. Suitable compositions can contain at least of one a protein, a carbohydrate, a fiber source, a mineral, a vitamin or other nutrient. The composition can be liquid or dry. A suitable protein source can be any suitable for ingestion by hogs, and thus, can be of plant, yeast or animal origin.

Thus, in one embodiment, the composition of interest is admixed with a food stuff or feed for finishing hogs. Thus, the composition of interest can be admixed to form a feed, or can be admixed with a feed. In some cases, the composition of interest can be added to a food, such as a plant, yeast or animal stuff, such as table scraps, remains, portions of foods unusable for sale, portions normally discarded in the food preparation business and so on. Because the composition of interest is relatively inert, the composition of interest can be admixed with any of a variety of food sources suitable for ingestion by a hog.

An alternative food or feed for hogs are prepared foods, such as a prepared, dry pelleted product. Such products are known in the art, sometimes known as a diet food or an animal feed, and the composition of each is a design choice. The composition of interest can be integrated into the feed during manufacturing and processing. Alternatively, the composition of interest can be added to the animal feed. For example, that can occur by admixing dry astaxanthin powder with the feed preparation. Alternatively, astaxanthin can be suspended in a liquid, such as a supplement containing a vitamin, a mineral or both, and administered to a hog, or can be admixed with a food or feed.

In a preferred embodiment of the instant invention, astaxanthin is used as an additive to a feed. However, other means of administration can be used. The astaxanthin can be added as part of a composition of two active agents, the other being Paylean®, or the two active ingredients can added separately, whether concurrently or sequentially.

The invention comprises a composition comprising astaxanthin and Paylean®. In another embodiment, the instant invention contemplates an article of manufacture, such as a kit containing as separate units, a first unit containing astaxanthin and a second unit containing Paylean®. By unit, the instant invention contemplates any means to contain an active, such as a vial, bottle, pouch, bag and so on, or any other packaging or enclosing means to contain a quantity of the actives. The kit also may include instructions for use.

The invention now will be exemplified in the following non-limiting example.

### Example 1

### Growth Performance and Carcass Characteristics of Pigs Fed Increasing Levels of Astaxanthin in Combination with Paylean®

Procedures used in the experiment were approved by the Kansas State University (KSU) Animal Care and Use Committee. The project was conducted at the KSU Swine Teaching and Research Farm. Pigs were housed in an environmentally-regulated finishing building with pens over a totally slatted floor that provided approximately 8 ft² per pig. Each pen was equipped with a dry, self-feeder and one nipple waterer, providing ad libitum access to feed and water. The facility was a mechanically-ventilated room with a pull-plug, manure storage pit.

A total of 144 finishing pigs from a single group (72 barrows and 72 gilts) were utilized for the experiments. Pigs were blocked by weight and gender, and allotted to one of 9 dietary treatments for an average period of 27 days pre-harvest. Each pen served as an experimental unit, with 2 pigs/pen and 8 replications to provide 16 pigs per treatment.

Experimental diets were provided in meal form and astaxanthin (0, 5, 7.5 and 10 ppm) was added to the control diet at the expense of corn starch to achieve the dietary treatments (Table 1). Paylean® was added to the diets indicated to contain 10 ppm.

Pigs were weighed on day 0, 7, 14, 21, and ˜27 to calculate average daily gain, average daily feed intake, and feed efficiency. Upon completion of the collection of growth performance data, all pigs were euthanized and processed for the collection of standard carcass measurements (dressing percentage, 10^{th} rib backfat depth, loin muscle area, etc.).

In the agricultural arts, certain carcass and meat parameters or characteristics are monitored for economic benefit, not only from the standpoint of feed conversion efficiency but also for consumer preference and appeal. Some of the characteristics of interest to a hog producer include ADG, average daily weight gain, ADFI, average daily feed intake, fat amounts, meat color and so on. Such carcass and meat characteristics can lead to improvements in the net profit/pig. The US Department of Agriculture provides for a grading of pork carcasses. Also, see, Buhr & DiPietre (1977) Front end guidance for value-added networks. National Pork Producers Council, Des Moines, Iowa; and National Pork Board (2000) Pork composition and quality assessment procedures. Berg, ed., Des Moines, Iowa.

Data were analyzed as a randomized complete block design using the PROC MIXED procedure of SAS with pen as the experimental unit. Linear and quadratic polynomial contrasts were used to determine the effect of increasing astaxanthin, with and without addition of Paylean®.

The source of natural astaxanthin used in this example was the yeast Phaffia rhodozyma which is commercially available under the trade name Aquasta® containing 10000 ppm astaxanthin, by weight.

TABLE 1
Composition of Experimental Diets

| | Added Dietary Astaxanthin, ppm | | | |
|---|---|---|---|---|
| | 0 | 5 | 7.5 | 10 |
| Ingredient, % | | | | |
| Corn | 85.40 | 85.40 | 85.40 | 85.40 |
| Soybean meal, 46.5% CP | 12.44 | 12.44 | 12.44 | 12.44 |
| Monocalcium P, 21% P | 0.45 | 0.45 | 0.45 | 0.45 |
| Limestone | 0.85 | 0.85 | 0.85 | 0.85 |
| Salt | 0.35 | 0.35 | 0.35 | 0.35 |
| L-lysine HCl | 0.15 | 0.15 | 0.15 | 0.15 |
| Vitamin premix | 0.08 | 0.08 | 0.08 | 0.08 |
| Trace mineral premix | 0.08 | 0.08 | 0.08 | 0.08 |
| Corn starch | 0.20 | 0.15 | 0.125 | .10 |
| Astaxanthin | — | 0.05 | 0.075 | 0.10 |
| (10,000 ppm) | | | | |
| TOTAL | 100.00 | 100.00 | 100.00 | 100.00 |
| Diet cost, $/100 pounds^{b} | 7.25 | 7.70 | 7.93 | 8.15 |
| Calculated analysis | | | | |
| Total lysine, % | 0.72 | 0.72 | 0.72 | 0.72 |
| True digestible amino | | | | |
| acids | | | | |
| Lysine, % | 0.63 | 0.63 | 0.63 | 0.63 |
| Isoleucine:lysine ratio, % | 71 | 71 | 71 | 71 |
| Leucine:lysine ratio, % | 188 | 188 | 188 | 188 |
| Methionine:lysine ratio, | 33 | 33 | 33 | 33 |
| % | | | | |
| Met & Cys:lysine ratio, | 68 | 68 | 68 | 68 |
| % | | | | |
| Threonine:lysine ratio, % | 64 | 64 | 64 | 64 |
| Tryptophan:lysine ratio, | 18 | 18 | 18 | 18 |
| % | | | | |
| Valine:lysine ratio, % | 85 | 85 | 85 | 85 |
| Protein, % | 13.2 | 13.2 | 13.2 | 13.2 |
| ME, kcal/lb | 1,522 | 1,522 | 1,522 | 1,522 |
| TID lysine:ME ratio, | 1.88 | 1.88 | 1.88 | 1.88 |
| g/Mcal | | | | |
| Ca, % | 0.47 | 0.47 | 0.47 | 0.47 |
| P, % | 0.42 | 0.42 | 0.42 | 0.42 |
| Available P, % | 0.15 | 0.15 | 0.15 | 0.15 |

TABLE 2

| | Added astaxanthin without | | | | | | | | | | | | | |
|---|---|---|---|---|---|---|---|---|---|---|---|---|---|---|
| | Paylean ® | | | | Added Aquasta ® astaxanthin | | | | | P< | | | | |
| | Aquasta ® | | | | with 10 ppm Paylean ® | | | | | | Aquasta ® Astaxanthin | | | |
| | 0 | 5 | 7.5 | 10 | 0 | 2.5 | 5 | 7.5 | | | −Paylean ® | | +Paylean ® | |
| | ppm | ppm | ppm | ppm | ppm | ppm | ppm | ppm | SEM | Paylean ® | linear | quad | linear | quad |
| Growth | | | | | | | | | | | | | | |
| performance, | | | | | | | | | | | | | | |
| 26 d | | | | | | | | | | | | | | |
| ADG, lb | 2.49 | 2.34 | 2.45 | 2.41 | 2.83 | 2.88 | 2.88 | 2.83 | 0.08 | 0.01 | — | — | — | — |
| ADFI, lb | 8.06 | 8.06 | 8.03 | 8.01 | 8.05 | 8.19 | 8.27 | 7.79 | 0.23 | — | — | — | — | — |
| F/G | 3.24 | 3.44 | 3.28 | 3.35 | 2.86 | 2.85 | 2.88 | 2.76 | 0.08 | 0.01 | — | — | — | — |
| G/F | 0.31 | 0.29 | 0.31 | 0.30 | 0.35 | 0.35 | 0.35 | 0.36 | 0.01 | 0.01 | — | — | — | — |
| Body | | | | | | | | | | | | | | |
| weight, lb | | | | | | | | | | | | | | |
| Initial | 225.6 | 225.7 | 225.3 | 225.7 | 225.6 | 225.4 | 225.5 | 225.3 | 4.90 | — | — | — | — | — |
| Final | 288.9 | 287.6 | 287.4 | 286.2 | 297.1 | 298.6 | 298.8 | 299.1 | 4.55 | 0.01 | — | — | — | — |
| Carcass | | | | | | | | | | | | | | |
| charact. | | | | | | | | | | | | | | |
| HCW, lb | 204.4 | 202.6 | 203.4 | 202.5 | 213.9 | 216.1 | 215.3 | 218.0 | 3.53 | 0.01 | — | — | — | — |
| Yield, % | 70.7 | 70.4 | 71.5 | 70.8 | 72.0 | 72.4 | 72.1 | 72.9 | 0.36 | 0.01 | — | — | — | — |
| BF-avg., | 1.02 | 1.12 | 1.02 | 1.04 | 1.06 | 1.06 | 1.05 | 1.03 | 0.03 | — | — | — | — | — |
| in. | | | | | | | | | | | | | | |
| BF-10^{th} | 0.78 | 0.84 | 0.81 | 0.77 | 0.81 | 0.78 | 0.82 | 0.70 | 0.05 | — | — | — | — | — |
| rib, in. | | | | | | | | | | | | | | |
| LEA- | 7.26 | 7.55 | 7.36 | 7.53 | 8.29 | 8.25 | 8.19 | 8.92 | 0.25 | 0.01 | — | — | 0.10 | 0.12 |
| 10^{th} rib, | | | | | | | | | | | | | | |
| in² | | | | | | | | | | | | | | |
| SFFL, lb | 108.4 | 107.1 | 107.6 | 108.6 | 115.2 | 116.8 | 115.4 | 121.5 | 2.43 | 0.01 | — | — | 0.05 | — |
| PFFL, % | 53.0 | 52.8 | 52.9 | 53.7 | 53.8 | 54.1 | 53.6 | 55.7 | 0.75 | — | — | — | — | — |

ADG = Average Daily Gain; ADFI = Average Daily Feed Intake; F/G = Feed Gain (Pounds of feed to gain one pound of weight); G/F = Gain Feed (Reciprocal of F/G); HCW = Hot Carcass Weight (Weight of harvested pig devoid of intestines, organs, head,, dehaired); BF = Back Fat (Measurement in (thickness) inches of fat); LEA = Loin Eye Area (Square inch measurement of boneless pork chop); SFFL = Standard Fat Free Lean (Weight in pounds of total edible muscle); and PFFL = Percent Fat Free Lean (SFFL/HCW).

As can be seen from the data in Table 2, pigs fed with 7.5 ppm astaxanthin alone had LEA (loin eye areas) which averaged 7.36 square inches as compared to 7.26 square inches for control pigs fed diets without astaxanthin, an increase of 0.10 square inches. Pigs feed with 10 ppm Paylean® alone had LEA values which averaged 8.29 square inches, an increase of 1.03 square inches over pigs fed the control diet without astaxanthin or Paylean®.

Surprisingly, when pigs were fed a combination of 7.5 ppm astaxanthin and 10 ppm Paylean®, LEA values averaged 8.92 square inches, an increase of 1.66 square inches over the pigs fed the control diet, and an increase of 0.63 square inches over pigs fed 10 ppm Paylean® alone, thus showing an unexpected improvement over the use of each active ingredient alone.

With respect to SFFL (standard fat free lean), pigs fed astaxanthin showed no statistical difference in SFFL over pigs fed a diet without astaxanthin. Pigs fed 10 ppm Paylean® showed a statistically significant increase in SFFL from 108.4 pounds to 115.2 pounds.

However, when pigs were fed with a diet containing 10 ppm Paylean® combined with 7.5 ppm astaxanthin from Aquasta® (which had no effect when used alone), the average SFFL increased to 121.5 pounds, an increase of 6.3 pounds over the treatment with 10 ppm Paylean® alone, again showing a statistically significant increase in a beneficial meat property over the use of each active ingredient alone.

All references cited herein, are herein incorporated by reference in entirety.

What is claimed is:

1. A composition comprising ractopamine and an astaxanthin.

2. The composition of claim 1, comprising a feed.

3. The composition of claim 2, wherein said ractopamine is present in an amount of 9 ppm.

4. The composition of claim 2, wherein said ractopamine is present in an amount of 10 ppm.

5. The composition of claim 3 or 4, wherein said astaxanthin in present in an amount of 7.5 ppm.

6. The composition of claim 1, wherein said astaxanthin is obtained from a yeast.

7. The composition of claim 6, wherein said yeast is Phaffia rhodozyma.

8. The composition of claim 7, wherein said Phaffia rhodozyma comprises at least 500 ppm astaxanthin per gram dry weight of yeast.

9. The composition of claim 8, wherein said Phaffia rhodozyma comprises at least 1000 ppm astaxanthin per gram dry weight of yeast.

10. An article of manufacture comprising astaxanthin and Paylean®, either separately or in combination, and optionally instructions for use.
